(19) United States
(12) Patent Application Publication
(10) Pub. No.: US 2020/0368078 A1
(43) Pub. Date: Nov. 26, 2020

(54) DISPOSABLE DIAPER FOR PET AND METHOD FOR MANUFACTURING DISPOSABLE DIAPER FOR PET

(71) Applicant: UNICHARM CORPORATION, Ehime (JP)

(72) Inventor: Daisuke Komatsubara, Kagawa (JP)

(73) Assignee: UNICHARM CORPORATION, Ehime (JP)

(21) Appl. No.: 16/878,992

(22) Filed: May 20, 2020

(30) Foreign Application Priority Data
May 20, 2019 (JP) 2019-094869
Nov. 11, 2019 (JP) 2019-203994

(51) Int. Cl. A61F 13/49; A61F 13/514; A61F 13/511

(52) U.S. Cl. CPC A61F 13/49061; A61F 13/49007; A61F 2013/15186; A61F 13/511; A61F 13/514

(57) ABSTRACT

A disposable diaper for a pet includes: a main body including a topsheet, a backsheet, and an absorbent core between the topsheet and the backsheet; and a binding portion that binds opposing parts of the main body when the disposable diaper is wrapped around a waist of the pet in a wearing state. The main body further includes: a main-body first end at one end in a waistline direction of the disposable diaper; a main-body second end at another end in the waistline direction opposite to the main-body first end and that is on an outside of the disposable diaper in the wearing state; and a mark portion, visible from a side of the backsheet, that indicates a position of the main-body second end in the wearing state.

### CROSS-REFERENCE TO RELATED APPLICATIONS

The entire contents of Japanese Patent Application No. 2019-094869 (filed on May 20, 2019) and Japanese Patent Application No. 2019-203994 (filed on Nov. 11, 2019) are incorporated herein by reference.

### TECHNICAL FIELD

The present invention relates to a disposable diaper for pet placed on a pet such as dog and cat.

### BACKGROUND

Patent Literatures 1 discloses disposable diapers for pet. The disposable diaper for pet disclosed in Patent Literature 1 a band-like main body having an absorbent core, an adhesive seal arranged on the top face side of the main body, and a seal receiving part arranged on the back face side of the main body. When the disposable diaper for pet disclosed in Patent Literature 1 is placed, the disposable diaper for pet is positioned so as to align the longitudinal direction of the main body to the waistline direction of a pet, and to cover the urethral opening of the pet with the absorbent core, thereby wrapping around the waist of the pet with the aid of the adhesive seal and the seal receiving part (see FIG. 3 of Patent Literature 1).

### PATENT LITERATURE

Patent Literature 1: JP 3141580U

However, in the disposable diaper for pet described in Patent Literature 1, the longitudinal direction of the main body of the disposable diaper for pet is laid around the waistline direction of the pet, and the transverse direction is disposed along the front-back direction that extends from the head side to the crotch side. Now, it is a general practice to put the disposable diaper for pet equally over the entire range of the front-back direction. The waist size of the pet, however, varies in the front-back direction. Hence, a gap has occasionally been produced between the waist of the pet and the disposable diaper, degrading the fitting quality.

### SUMMARY

One or more embodiments of the present invention provide a disposable diaper for pet that can be placed conforming to the waist shape of pet, and can improve the fitting quality.

A disposable diaper for pet according to one or more embodiments includes: a waistline direction disposed along a waistline direction of a pet; a cross direction orthogonal to the waistline direction; a main body portion (main body) having a topsheet, a backsheet, and an absorbent core arranged between the topsheet and the backsheet; and a binding portion that binds opposing parts of the main body portion when wrapped around a waist of the pet in a wearing state. The main body portion includes: a main-body first end which forms one end in the waistline direction; a main-body second end which forms the other end in the waistline direction, and arranged an outside of the disposable diaper in the wearing state; a main-body third end which forms one end in the cross direction, and arranged on a head-side of the pet in the wearing state; a main-body fourth end which forms the other end in the cross direction, and arranged on a crotch-side of the pet in the wearing state; and a mark portion being designed visible from a side of the backsheet of the disposable diaper for pet, and to indicate a position of the main-body second end in the wearing state. The mark portion includes: a head-side mark portion residing on a side of the main-body third end of a center in the cross direction of the main body portion (i.e., a head-side mark portion disposed on one side from a center of the disposable diaper in the cross direction toward the main-body third end); and a crotch-side mark portion residing on a side of the main-body fourth end of the center in the cross direction of the main body (i.e., a crotch-side mark portion disposed on another side from the center of the disposable diaper in the cross direction toward the main-body fourth end). The head-side mark portion is positioned inside, in the waistline direction, of the crotch-side mark portion.

A disposable diaper for pet according to one or more embodiments includes: a waistline direction disposed along a waistline direction of a pet; a cross direction orthogonal to the waistline direction; a main body portion having a topsheet, a backsheet, and an absorbent core arranged between the topsheet and the backsheet; and a binding portion that binds opposing parts of the main body portion when wrapped around a waist of the pet in a wearing state. The main body portion includes: a main-body first end which forms one end in the waistline direction; a main-body second end which forms the other end in the waistline direction, and arranged an outside of the disposable diaper in the wearing state; a main-body third end which forms one end in the cross direction, and arranged on a head-side of the pet in the wearing state; a main-body fourth end which forms the other end in the cross direction, and arranged on a crotch-side of the pet in the wearing state; and a mark portion being designed visible from a side of the backsheet of the disposable diaper for pet, and to indicate a position of the main-body second end in the wearing state. The mark portion includes: a head-side mark portion residing on a side of the main-body third end of a center in the cross direction of the main body portion; and a crotch-side mark portion residing on a side of the main-body fourth end of the center in the cross direction of the main body. The head-side mark portion is positioned outside, in the waistline direction, of the crotch-side mark portion.

### BRIEF DESCRIPTION OF DRAWINGS

FIG. 1 is a plan view in which a disposable diaper according to one or more embodiments of the present invention is seen from a skin contact surface side.

FIG. 2 is a plan view illustrating the disposable diaper for pet of one or more embodiments, when viewed from the non-skin contact side.

FIG. 3 is a schematic cross sectional view of the disposable diaper for pet taken along the line A-A in FIG. 1.

FIG. 4 is a drawing schematically illustrating a wearing state of the disposable diaper for pet.

FIG. 5 is a plan view illustrating the disposable diaper for pet of one or more embodiments, when viewed from the skin contact side.

FIG. 6 is a drawing schematically illustrating a wearing state of the disposable diaper for pet of one or more embodiments.

FIGS. 7(a) to 7(c) are plan views illustrating the disposable diaper for pet of modified examples 1 to 3, when viewed from the non-skin contact side.

FIGS. 8(a) to 8(c) are plan views illustrating the disposable diaper for pet of modified examples 4 to 6, when viewed from the non-skin contact side.

FIGS. 9(a) to 9(c) are plan views illustrating the disposable diaper for pet of modified examples 7 to 9, when viewed from the non-skin contact side.

FIGS. 10(a) to 10(c) are plan views illustrating the disposable diaper for pet of modified examples 10 to 12, when viewed from the non-skin contact side.

### DETAILED DESCRIPTION

### (1) Outline of Embodiments

According to the present specification and the accompanying drawings, at least the following matters will be disclosed.

A disposable diaper for pet according to one or more embodiments includes: a waistline direction disposed along a waistline direction of a pet; a cross direction orthogonal to the waistline direction; a main body portion having a topsheet, a backsheet, and an absorbent core arranged between the topsheet and the backsheet; and a binding portion that binds opposing parts of the main body portion when wrapped around a waist of the pet in a wearing state. The main body portion includes: a main-body first end which forms one end in the waistline direction; a main-body second end which forms the other end in the waistline direction, and arranged an outside of the disposable diaper in the wearing state; a main-body third end which forms one end in the cross direction, and arranged on a head-side of the pet in the wearing state; a main-body fourth end which forms the other end in the cross direction, and arranged on a crotch-side of the pet in the wearing state; and a mark portion being designed visible from a side of the backsheet of the disposable diaper for pet, and to indicate a position of the main-body second end in the wearing state. The mark portion includes: a head-side mark portion residing on a side of the main-body third end of a center in the cross direction of the main body portion; and a crotch-side mark portion residing on a side of the main-body fourth end of the center in the cross direction of the main body. The head-side mark portion is positioned inside, in the waistline direction, of the crotch-side mark portion. According to one or more embodiments, the user can align the main-body second end in the wearing state, referring to the mark portion. More specifically, the user places the main body portion around the waist of pet while aligning the waistline direction of the main body portion along the waist, and binds parts of the main body portion by the binding portion. Some sort of pet may have the head-side size shorter than the crotch-side size. With the head-side mark portion residing inside, in the waistline direction, of the crotch-side mark portion, and as a result of placement, by the user, of the main-body second end along the head-side mark portion and the crotch-side mark portion, the disposable diaper can be placed so as to make the head-side size shorter than the crotch-side size. The disposable diaper can therefore be placed conforming to the waist shape of pet, and can improve the fitting quality.

A disposable diaper for pet according to one or more embodiments includes: a waistline direction disposed along a waistline direction of a pet; a cross direction orthogonal to the waistline direction; a main body portion having a topsheet, a backsheet, and an absorbent core arranged between the topsheet and the backsheet; and a binding portion that binds opposing parts of the main body portion when wrapped around a waist of the pet in a wearing state. The main body portion includes: a main-body first end which forms one end in the waistline direction; a main-body second end which forms the other end in the waistline direction, and arranged an outside of the disposable diaper in the wearing state; a main-body third end which forms one end in the cross direction, and arranged on a head-side of the pet in the wearing state; a main-body fourth end which forms the other end in the cross direction, and arranged on a crotch-side of the pet in the wearing state; and a mark portion being designed visible from a side of the backsheet of the disposable diaper for pet, and to indicate a position of the main-body second end in the wearing state. The mark portion includes: a head-side mark portion residing on a side of the main-body third end of a center in the cross direction of the main body portion; and a crotch-side mark portion residing on a side of the main-body fourth end of the center in the cross direction of the main body. The head-side mark portion is positioned outside, in the waistline direction, of the crotch-side mark portion. According to one or more embodiments, the user can align the main-body second end in the wearing state, referring to the mark portion. More specifically, the user places the main body portion around the waist of pet while aligning the waistline direction of the main body portion along the waist, and binds parts of the main body portion by the binding portion. Some sort of pet may have the head-side size longer than the crotch-side size. With the head-side mark portion positioned outside, in the waistline direction, of the crotch-side mark portion, and as a result of placement, by the user, of the main-body second end along the head-side mark portion and the crotch-side mark portion, the disposable diaper can be placed so as to make the head-side size longer than the crotch-side size. The disposable diaper can therefore be placed conforming to the waist shape of pet, and can improve the fitting quality.

According to one or more embodiments, the binding portion is arranged on a face of a backsheet side of the main-body first end, and bound to a face of a top sheet side of the main-body second end in the wearing state. At least either the head-side mark portion or crotch-side mark portion is arranged (i.e., one of the head-side mark portion or the crotch-mark portion is arranged) in a region adjacent to the binding portion. According one or more embodiments, the user can easily recognize the mark portion when handling the binding portion, and will become more aware of presence of the mark portion. Hence, the disposable diaper can therefore be placed conforming to the waist shape of pet, referring to the mark portion.

According to one or more embodiments, the binding portion is arranged on a face of a backsheet side of the main-body first end, and bound to a side of a top sheet side of the main-body second end in the wearing state. At least either the head-side mark portion or crotch-side mark portion is arranged in a region overlapping the binding portion. According to one or more embodiments, the user can easily recognize the mark portion when handling the binding portion, and can place the disposable diaper conforming to the waist shape of pet, referring to the mark portion.

According to one or more embodiments, at least either the head-side mark portion or the crotch-side mark portion indicates a position of an outer edge in the waistline direction of the main-body second end in the wearing state. According to one or more embodiments, the user can intuitively recognize the position and angle of the outer edge in the waistline direction of the main-body second end in the wearing state. By aligning the outer edge in the waistline direction of the main-body second end to the mark portion, the user can place the disposable diaper at a proper position and angle.

According to one or more embodiments, the binding portion is arranged on the main-body first end. The mark portion is formed of the binding portion. When binding the main-body second end to the binding portion for placement according to one or more embodiments, it is a general practice to align the main-body second end along the binding portion. By aligning the main-body second end along the binding portion, the user can place the disposable diaper conforming to the waist shape of pet unconsciously.

According to one or more embodiments, the binding portion has a head-side binding portion that composes the head-side mark portion, and a crotch-side binding portion that composes the crotch-side mark portion. The head-side binding portion and the crotch-side binding portion are arranged in the waistline direction with a space in between (i.e., spaced apart in the waistline direction). According to one or more embodiments, with the head-side binding portion and the crotch-side binding portion arranged with a space in between, the user can stepwise bind the main-body second end, as compared with a structure having the head-side binding portion and the crotch-side binding portion integrated therein. Hence, even women or children with small hands can easily place the disposable diaper just as they intended, and can improve the fitting quality.

According to one or more embodiments, the head-side mark portion and the crotch-side mark portion are connected and extend in the cross direction, obliquely to the cross direction. With the head-side mark portion and the crotch-side mark portion connected, one or more embodiments enable sequential alignment of the main-body second end, and gradual binding of the main-body second end to the binding portion. Hence, even women or children with small hands can easily place the disposable diaper just as they intended, and can improve the fitting quality.

According to one or more embodiments, the mark portion is formed of an outer edge on a side of the main-body first end of the absorbent core (i.e., an outer edge of the absorbent core on a side of the absorbent core closer to the main-body first end). One or more embodiments may also provide guidance to a placement position of the main-body second end, with an improved decorativeness given by the printed part.

According to one or more embodiments, the mark portion may be formed of the outer edge on the main-body first end side of the absorbent core. According to one or more embodiments, the user will become easier to recognize a position of the outer edge of the absorbent core, in the process of alignment referring to the mark portion for placement. By recognizing the position of the outer edge of the absorbent core, the user can dispose the absorbent core so as not to cause leakage.

A method for manufacturing disposable diaper for pet according to one or more embodiments includes a sheet feeding step of feeding a continuous sheet having sheet components arrayed; a marking step of giving (e.g., disposing by printing or embossing) a mark portion on the continuous sheet; and a core laminating step of laminating an absorbent core on the continuous sheet having the mark portion. The core laminating step being designed to detect a position of the mark portion on the continuous sheet, and to adjust a position of lamination of the absorbent core referring to the position of the mark portion.

With present method for manufacturing, the positional relation between the mark portion and the absorbent core may be kept constant. Hence, the disposable diaper when placed referring to the mark portion will have the absorbent core whose placement position is prevented from being misaligned one by one. With suppression of shift of the mark portion over the whole range of the disposable diaper, alignment referring to such mark portion will be enabled with improved accuracy.

### (2) Overall Structure of Disposable Diaper for Pet

A disposable diaper for pet according to one or more embodiments will be described below by referring to the accompanying drawings. In the drawings, the same or similar parts are indicated by the same or similar reference signs. The drawings are illustrated schematically, and dimensional ratio and other variables differ from those of actual measurements. The actual measurements or the like, therefore, should be determined by referring to the following description. The drawings may include different relationships or ratios of measurements.

In this patent specification, “pet” widely encompasses vertebrate and invertebrate animals, and typically include pet animals such as cat, dog, rabbit and hamster. The disposable diaper in one or more embodiments is a disposable diaper for dog. In a modified example, the disposable diaper may be the one for cat.

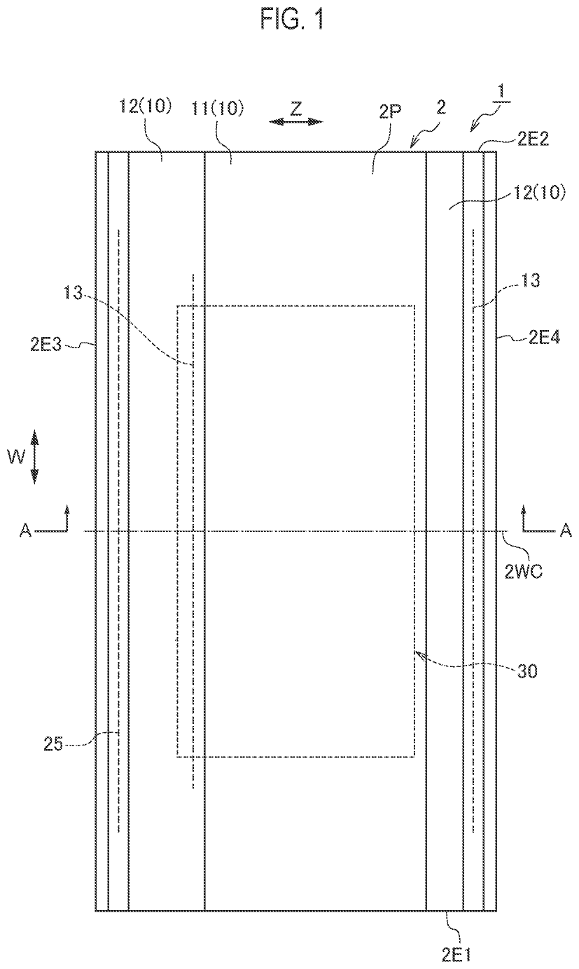

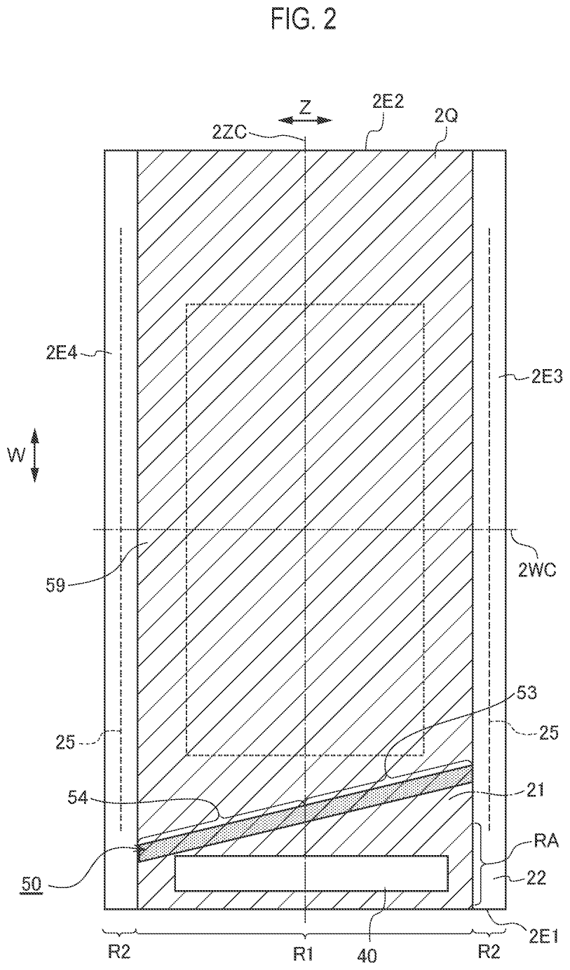

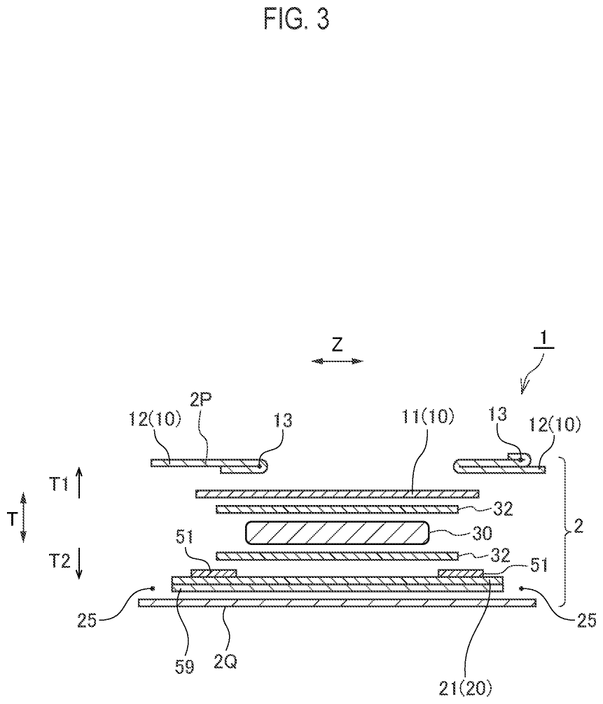

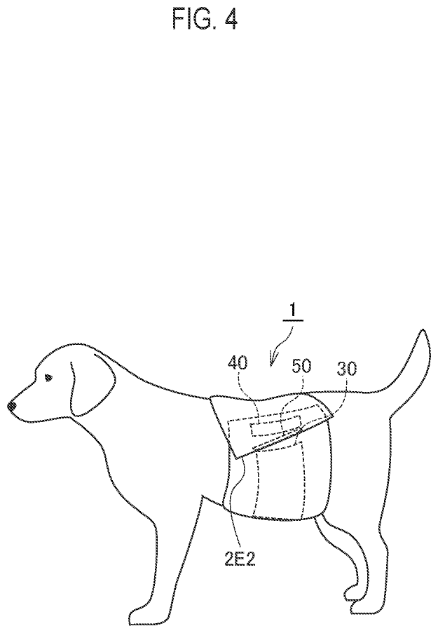

FIG. 1 is a plan view illustrating the disposable diaper for pet of one or more embodiments, when viewed from the skin contact side. FIG. 2 is a plan view illustrating the disposable diaper for pet of one or more embodiments, when viewed from the non-skin contact side. FIG. 3 is a schematic cross sectional view of the disposable diaper for pet taken along the line A-A in FIG. 1. FIGS. 1 and 2 illustrate a disposable diaper for pet 1, extended until any wrinkle will disappear. The individual members, although illustrated in the cross sectional view of FIG. 3 as if they are apart from each other in the thickness direction T for the convenience of explanation, are brought into close contact in the thickness direction T in the actual product. FIG. 4 is a drawing schematically illustrating a wearing state of the disposable diaper for pet.

The disposable diaper for pet has waistline direction W laid in the waistline direction of the pet, cross direction Z laid orthogonal to the waistline direction W, and the thickness direction T laid orthogonal to the waistline direction W and to the cross direction Z. The cross direction Z is laid in the longitudinal direction of the pet, that is, the direction connecting the crotch-side with the far side of the crotch. The thickness direction T is laid towards the top face side T1 faced to the pet in the wearing state, and towards the back face side T2 faced outside in the wearing state. As illustrated in FIG. 4, the disposable diaper for pet of one or more embodiments is placed so as to wrap around the waist of the pet, rather than being placed so as to cover the pet from the belly through the crotch to the back. Hence the length in the waistline direction W of the disposable diaper for pet 1 is longer than the length in the cross direction Z of the disposable diaper for pet 1.

The disposable diaper for pet 1 has a main body portion 2 and a binding portion 40. The main body portion 2 may have a main-body first end 2E1 which forms one end in the waistline direction W, a main-body second end 2E2 which forms the other end in the waistline direction W, a main-body third end 2E3 which forms one end in the cross direction Z, and a main-body fourth end 2E4 which forms the other end in the cross direction Z. The main-body first end 2E1, when placed around the waist of pet in the wearing state (see FIG. 4) is covered with the main-body second end 2E2, and positioned inside (pet side) of the disposable diaper. The main-body second end 2E2 is opposed to the main-body first end 2E1, and is disposed on the outside of the disposable diaper in the wearing state. The main-body third end 2E3 is disposed on the head-side of the pet in the wearing state. The main-body fourth end 2E4 is opposed to the main-body third end 2E3, and is disposed on the crotch side (hip side) of the pet in the wearing state.

The main body portion 2 has at least a topsheet 10, a backsheet 20, and an absorbent core 30. The topsheet 10 is arranged on an inner face 2P of the main body portion 2, which is brought into contact with the pet. The topsheet 10 has liquid permeability that allows body fluid to permeate towards the absorbent core 30. The topsheet 10 may have a center sheet 11 arranged at the center in the cross direction Z so as to cover the absorbent core 30, and side sheets 12 individually covering both side part in the cross direction Z of the center sheet 11. The topsheet 10 may have thereon a printed part. The printed part may form a mark portion 50 described later.

The side sheets 12 may be folded as illustrated in FIG. 3. More specifically, the side sheet 12 on a main-body third end 2E3 side (left side in FIG. 1) is folded back at the inner end of the side sheet 12 towards the back face side T2. Meanwhile, the side sheet 12 on a main-body fourth end 2E4 side (right side in FIG. 1) is folded back at the inner end of the side sheet 12 towards the top face side T1, and the thus folded part is again folded back towards the top face side T1. A side elastic member 13 while being stretched in the waistline direction W is arranged in between folded parts of the side sheets 12. The side elastic member 13 may form leakproof gathers that rise up towards the pet. In the wearing state, one end side in the cross direction Z may be arranged on the front side of the pet, and the other end side in the cross direction Z may be arranged on the back side the pet in the wearing state. The side sheet on one end side in the cross direction Z and the side sheet on the other end side in the cross direction, although being asymmetric in one or more embodiments, may be symmetric in a modified example. The end in the context of one or more embodiments of the present invention is defined as a part that occupies a certain range including the edge.

The absorbent core 30 is arranged between the topsheet 10 and the backsheet 20. The absorbent core 30 is made of a stack of pulp or other absorption materials. The absorbent core 30 may have a core wrap 32 that covers the absorbent core 30. The absorbent core 30 is arranged, in the waistline direction W, inside of both edges 2WE of the main body portion 2. That is, the length in the waistline direction W of the absorbent core 30 is shorter than the length in the waistline direction W of the main body portion 2. The absorbent core 30 is arranged at the center in the waistline direction W of the main body portion 2, but is not arranged outside in the waistline direction W of the main body portion 2. The absorbent core 30 may be arranged, in the cross direction Z, inside of both edges 2ZE of the main body portion 2. That is, the length in the cross direction Z of the absorbent core 30 may be shorter than the length in the cross direction Z of the main body portion 2. The absorbent core 30 may be arranged at the center in the cross direction Z of the main body portion 2, but is not necessarily arranged outside in the cross direction Z of the main body portion 2. The absorbent core 30 and the core wrap 32 may have arranged thereto a printed part. The printed part may form a mark portion 50.

The backsheet 20 is arranged on an outer face 2Q of the main body portion 2, which appears on the outside in the wearing state. The backsheet 20 may have a non-liquid permeable back film 21 and a nonwoven back fabric 22 arranged on the outer side of the back film 21. In a modified example, the backsheet may have a non-liquid permeable back film and a nonwoven back fabric arranged on the inner face side of the back film 21. The back film 21 may have, on a face thereof on the top face side T1, an indicator that causes a coloring reaction upon contact with body fluid, and a printed part, meanwhile the back film 21 may have, on a face thereof on the back face side T2, a printed part. At least one of the indicator or the printed part (i.e., the indicator and/or the printed part alone) may form the mark portion 50. That is, the length in the cross direction Z of the back film 21 may be shorter than the length in the cross direction Z of the nonwoven back fabric 22. That is, the nonwoven back fabric 22 may extend on both sides in the cross direction Z out from the back film 21. In one or more embodiments where the printed part is arranged entirely over the back film 21, an area where the back film 21 is arranged may form a printed area R1, and a non-printed area R2 free of printing may be arranged on both sides in the cross direction Z of the back film 21.

The main body portion 2 may have, on both ends thereof in the cross direction Z, waistline gathers that stretch and contract in the waistline direction W. The waistline gathers are positioned outside in the cross direction Z of the absorbent core 30, and may be composed of the topsheet 10, the backsheet 20, and a waistline elastic member 25. The waistline elastic member 25 may be arranged between the topsheet 10 and the backsheet 20, and may be fixed, while being stretched in the waistline direction W, to the topsheet 10 and the backsheet 20. The waistline elastic member 25 and the aforementioned side elastic member 13 compose the elastic member in one or more embodiments of the present invention.

The binding portion 40, in the wearing state, binds the opposing parts of the main body portion 2. More specifically, the binding portion 40 is a binding means that binds the outer face 2Q of the main-body first end 2E1 to the inner face 2P of the main-body second end 2E2. The binding portion 40 in one or more embodiments is arranged on the main-body first end 2E1 of the main body portion 2. In more detail, the binding portion 40 is a mechanical fastener arranged on the outer face of the main-body first end 2E1. The binding portion 40 is made so as to be bound on the topsheet 10 of the main-body second end 2E2 of the main body portion 2. The disposable diaper for pet 1 may be placed on the pet, by placing it so as to cover the binding portion 40 with the main-body second end 2E2, by which the binding portion 40 is bound on the topsheet 10 of the main-body second end 2E2, and can keep the wearing state of the disposable diaper for pet. In a modified example, the binding portion 40 may be arranged on the inner face 2P of the main-body second end 2E2, so as to be bound on the backsheet 20 of the main-body first end 2E1. That is, in the modified example, the binding portion 40 may be arranged on the inner face 2P of the main body portion 2, so as to be bound on the backsheet of the main body portion 2.

Next, the mark portion 50 will be explained. The mark portion 50 provides guidance to a placement position of the disposable diaper for pet 1. The mark portion 50 is made visually recognizable from the backsheet side (outer face side) of the disposable diaper for pet 1. The backsheet side of the disposable diaper for pet, when placed, appears on the outer side and is visually recognizable after placing the disposable diaper to the pet, enabling positional adjustment not only in the process of placement, but also after placement. Even if the disposable diaper should turn in the waistline direction to cause displacement during wearing, such displacement is recognizable referring to the mark portion, enabling repositioning to the suitable position.

Now “visually recognizable” in the context of one or more embodiments of the present invention means that a subject with healthy vision in both eyes (20/20 vision or higher) can recognize an object from approximately 30 to 50 cm away, in a bright (approximately 500 to 750 lx) room with daylight illumination (color temperature ranging from 4600 to 5400 K).

The mark portion 50 indicates a position of the main-body second end 2E2 in the wearing state. The mark portion 50 in the wearing state is arranged in an area that overlaps the main-body second end 2E2, so that the user can dispose the main-body second end 2E2 so as to make it overlap the mark portion 50. In one or more embodiments, the mark portion 50 may be an arrow that points a position of the main-body second end 2E2 in the wearing state, and the user may dispose the main-body second end 2E2 at the position pointed by the mark portion 50. The mark portion 50 in one or more embodiments indicates a position of the edge of the main body portion 2 in the wearing state in the main-body second end 2E2.

The mark portion 50 has a head-side mark portion 53 that resides on the main-body third end 2E3 side of the center 2ZC in the cross direction Z of the main body portion 2, and a crotch-side mark portion 54 that resides on the main-body fourth end 2E4 side of the center 2ZC in the cross direction Z of the main body portion 2. The head-side mark portion 53 and the crotch-side mark portion 54 may separate from each other, or may be integrated. In a mode where the head-side mark portion 53 and the crotch-side mark portion 54 are integrated, the boundary between the head-side mark portion 53 and the crotch-side mark portion 54 may lie on the center 2ZC in the cross direction Z of the main body portion 2. The head-side mark portion 53 resides inside, in the waistline direction W, of the crotch-side mark portion 54. The inside in the waistline direction W means the center 2WC side in the waistline direction W of the main body portion 2. Hence, at least a part of the head-side mark portion 53 resides on the center 2WC side in the waistline direction W of the main body portion 2, than the crotch-side mark portion 54 does.

The waistline direction W of the main body portion 2 is laid around the waistline direction of the pet, meanwhile the cross direction Z of the main body portion 2 is disposed along the front-back direction that extends from the head side to the crotch side. The waist size of pet varies in the front-back direction, and some pet has a waist size shorter on the head side, than on the crotch side. Hence, a disposable diaper for pet having a rectangular main body portion 2 in plan view has occasionally produced a gap between the waist of pet and the disposable diaper, degrading the fitting quality. Waist size of pet varies depending on species of pet, and also changes depending on seasons due to molting. Hence the user may feel it difficult to place the disposable diaper well conforming to the shape of pet body.

In contrast, according to one or more embodiments, the user can place the disposable diaper so as to make the head-side size shorter than the crotch-side size, by aligning a position in the wearing state of the main-body second end 2E2, referring to the mark portion 50. More specifically, as illustrated in FIG. 4, the user places the main body portion 2 around the waist of pet while aligning the waistline direction W of the main body portion 2 along the waist, and binds parts of the main body portion 2 with the binding portion 40. With the head-side mark portion 53 residing inside, in the waistline direction W, of the crotch-side mark portion 54, and as a result of placement of the main-body second end 2E2 along the head-side mark portion 53 and the crotch-side mark portion 54, the disposable diaper can be placed so as to make the head-side size shorter than the crotch-side size. Placement well conforming to the shape of the waist of pet can improve the fitting quality.

The binding portion 40 in one or more embodiments is arranged on a face on the backsheet side of the main-body first end 2E1, and is formed so as to be bound with a face on the topsheet side of the main-body second end 2E2 in the wearing state. At least either the head-side mark portion 53 or the crotch-side mark portion 54 may be arranged in an adjoining area RA that adjoins the binding portion 40. The adjoining area RA is a region around the binding portion 40 in plan view, and may be an area within 20 mm from the binding portion 40. FIG. 2 illustrates a range in the waistline direction of the adjoining area RA. The user, handling the disposable diaper for pet 1 for placement, can recognize not only the binding portion 40, but also the adjoining area RA around it. Hence the user will become more aware of presence of the mark portion 50, and can place the disposable diaper conforming to the waist shape of pet, referring to the mark portion 50.

At least either the head-side mark portion 53 or the crotch-side mark portion 54 may point the outer edge in the waistline direction W of the main-body second end 2E2 in the wearing state. The user can intuitively recognize the position and angle of the outer edge in the waistline direction W of the main-body second end 2E2 in the wearing state. By aligning the outer edge in the waistline direction W of the main-body second end 2E2 to the mark portion 50, the user can place the disposable diaper at a proper position and angle.

The head-side mark portion 53 and the crotch-side mark portion 54 may be connected, and may extend in the cross direction Z, obliquely to the cross direction Z. With the head-side mark portion 53 and the crotch-side mark portion 54 connected, the main-body second end 2E2 may be aligned sequentially. The user can therefore recognize the mark portion 50, and can gradually bind the main-body second end 2E2 with the binding portion 40, enabling easy placement at a proper position.

The mark portion 50 may be a printed part arranged on the backsheet 20. The printed part can take part in guidance to a placement position of the main-body second end 2E2, while enhancing the decorativeness. The back film 21 of one or more embodiments has the printed part arranged over the entire range of the outer face. The printed part may form the mark portion 50, as well as the decoration part 59. As illustrated in FIG. 2, the back film in one or more embodiments has, on the outer face thereof, a printed part that forms the mark portion 50, and a printed part that forms a decoration part 59. The decoration part 59 is a part having design other than the mark portion, and has not only picture or pattern, but also background color solely represented by color. In a modified example, the mark portion may be an indicator arranged on the backsheet 20 or the core wrap 32.

In one or more embodiments, the mark portion 50 may be a boundary mark portion. More specifically, the printed part may have a first decoration part, and a second decoration part designed differently from the first decoration part, and the boundary between the first decoration part and the second decoration part may form a boundary mark portion. One or more embodiments enable alignment referring to the mark portion 50, while enjoying improved decorativeness given by two designs of the first decoration part and the second decoration part.

The backsheet 20 may have, as illustrated in FIG. 2, the printed area R1 having printed therein a design including the mark portion 50, and the non-printed area R2 free of printing. The non-printed areas R2 may be arranged at least on both ends in the cross direction Z of the backsheet 20. The user can recognize both ends in the cross direction Z of the main body portion 2, referring to a boundary between the non-printed area R2 and the printed area R1. Provision of the non-printed area R2 makes the printed area R1 more distinctive, making the mark portion 50 more easily recognizable in the printed area R1. The boundary between the non-printed area R2 and the printed area R1 may extend in the waistline direction W, and may reside outside in the cross direction Z of the absorbent core 30. That is, the non-printed area R2 may be arranged only on the outer side in the cross direction Z of the absorbent core 30. The user can recognize an area where the absorbent core 30 is not arranged, referring to the non-printed area R2, and can thereby position the absorbent core 30 so as not to cause leakage.

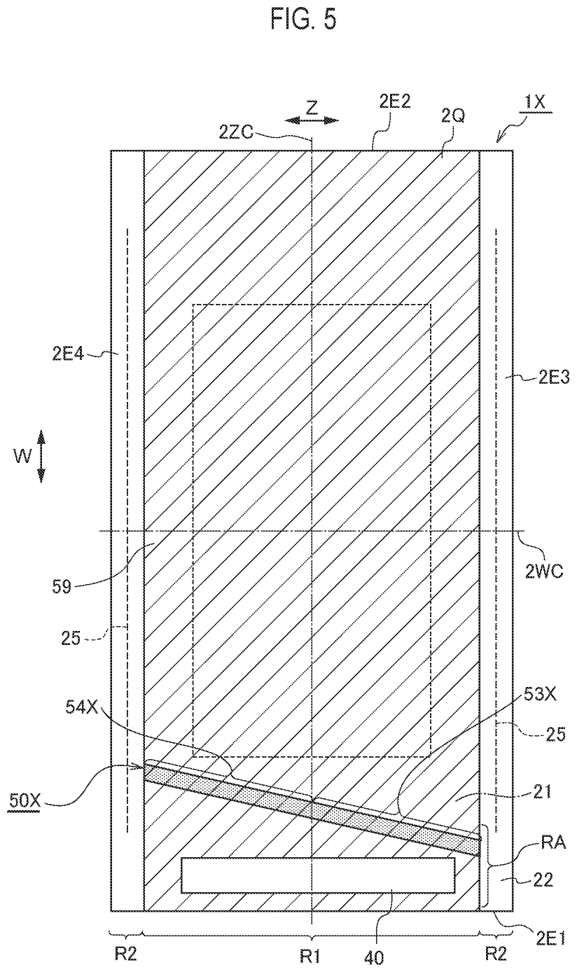

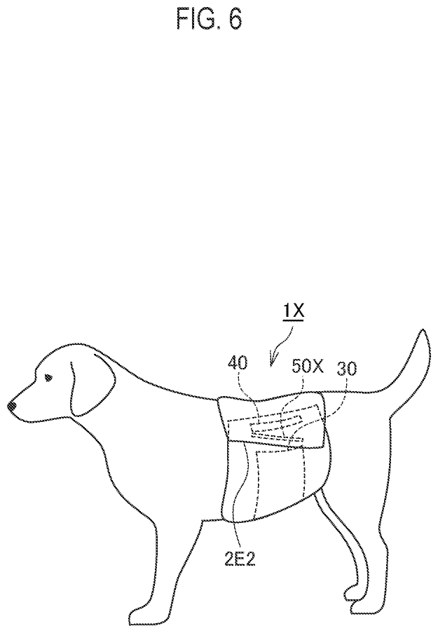

Next, a disposable diaper for pet 1X of one or more embodiments will be explained, referring to FIGS. 5 and 6. FIG. 5 is a plan view illustrating the disposable diaper for pet of one or more embodiments, when viewed from the non-skin contact side. FIG. 6 is a drawing schematically illustrating the wearing state of the disposable diaper for pet of one or more embodiments. Note that all structures same as those in the aforementioned embodiments will be given the same reference signs to avoid redundant explanation. The mark portion 50X of the disposable diaper for pet 1X of one or more embodiments has a head-side mark portion 53X and a crotch-side mark portion 54X. The head-side mark portion 53X resides outside, in the waistline direction W, of the crotch-side mark portion 54X. Hence, at least a part of the crotch-side mark portion 54X resides on the center 2WC side in the waistline direction W of the main body portion 2, than the head-side mark portion 53X does.

The waistline direction W of the main body portion 2 is laid around the waistline direction of the pet, meanwhile the cross direction Z of the main body portion 2 is disposed along the front-back direction that extends from the head side to the crotch side. The waist size of the pet varies in the front-back direction, and some pet has a waist size longer on the head side, than on the crotch side. Hence, a disposable diaper for pet having a rectangular main body portion 2 in plan view has occasionally produced a gap between the waist of pet and the disposable diaper, degrading the fitting quality. Waist size of pet varies depending on species of pet, and also changes depending on seasons due to molting. Hence the user may feel it difficult to place the disposable diaper well conforming to the shape of pet body.

In contrast, according to one or more embodiments, the user can place the disposable diaper so as to make the head-side size longer than the crotch-side size, by aligning a position in the wearing state of the main-body second end 2E2, referring to the mark portion 50X. More specifically, as illustrated in FIG. 6, the user places the main body portion 2 around the waist of pet while aligning the waistline direction W of the main body portion 2 along the waist, and binds parts of the main body portion 2 with the binding portion 40. With the head-side mark portion 53X residing outside, in the waistline direction W, of the crotch-side mark portion 54X, and as a result of placement of the main-body second end 2E2 along the head-side mark portion 53X and the crotch-side mark portion 54X, the disposable diaper can be placed so as to make the head-side size longer than the crotch-side size. Placement well conforming to the shape of the waist of pet can improve the fitting quality.

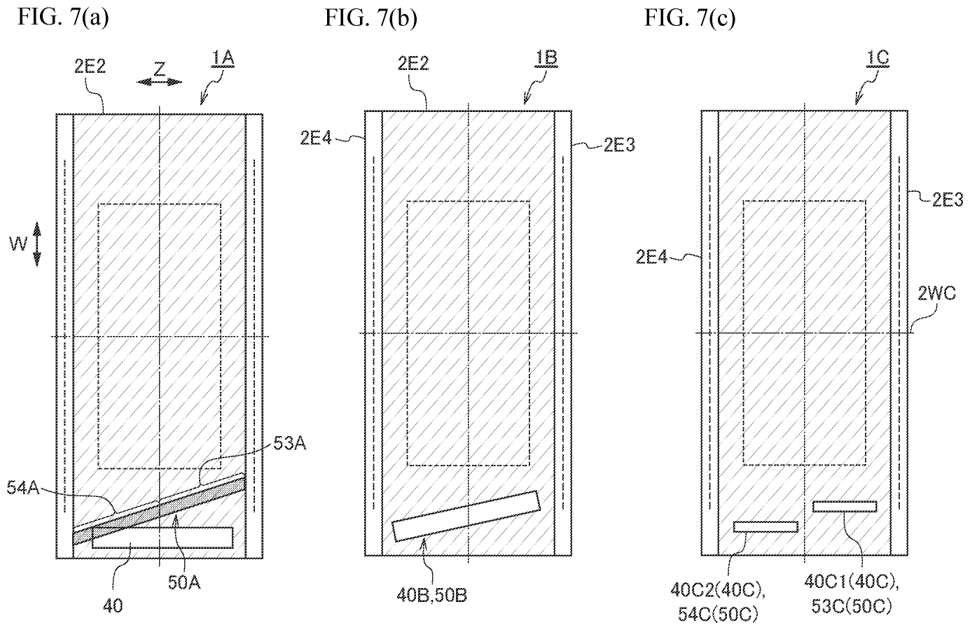

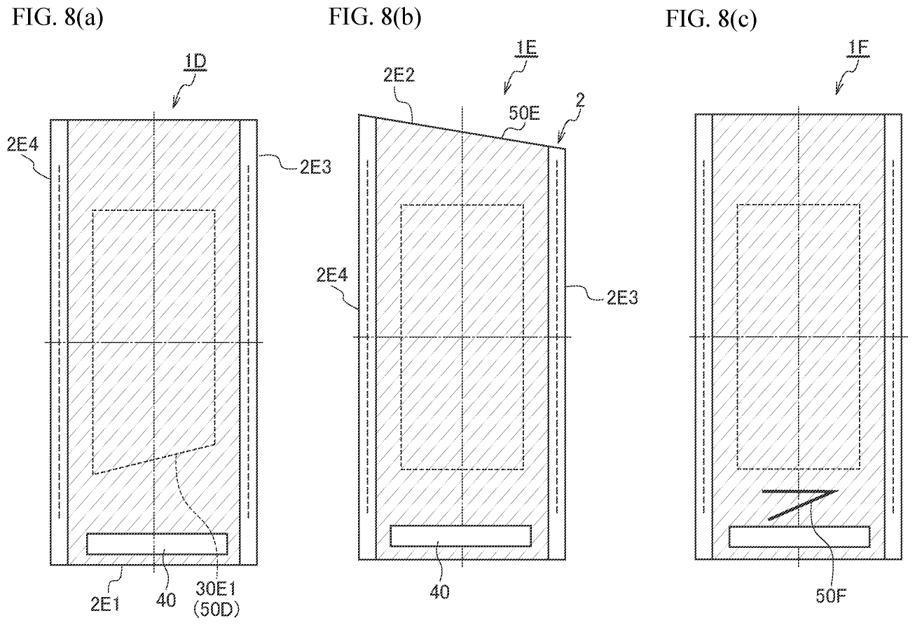

Next, a disposable diaper for pet of modified examples will be explained. Note that all structures same as those in the aforementioned embodiments will be given the same reference signs to avoid redundant explanation. FIGS. 7(a) to 7(c) and 8(a) to 8(c) are plan views illustrating modified examples of the disposable diaper for pet of one or more embodiments, viewed from the non-skin contact side of the disposable diaper for pet.

FIG. 7(a) illustrates a disposable diaper for pet 1A of Modified Example 1. The disposable diaper for pet 1A of modified example 1 has a mark portion 50A in which at least either a head-side mark portion 53A or a crotch-side mark portion 54A is arranged in an area that overlaps the binding portion 40. More specifically, the crotch-side mark portion 54A is arranged in the area that overlaps the binding portion 40. Note that at least a part of the crotch-side mark portion 54A may overlap the binding portion 40, or at least a part of the head-side mark portion 53A may overlap the binding portion 40. The user can easily recognize the mark portion 50A when handling the binding portion. The disposable diaper for pet 1A of modified example 1 may easily be placed conforming to the waist shape of pet, referring to the mark portion 50A.

FIG. 7(b) illustrates a disposable diaper for pet 1B of modified example 2. A mark portion 50B of a disposable diaper for pet 1B of modified example 2 is formed of a binding portion 40B. The edge in the waistline direction of the binding portion 40B in modified example 2 inclines towards the inside in the waistline direction, when viewed from the main-body fourth end 2E4 towards the main-body third end 2E3. The binding portion 40B has a rectangular shape in plan view. The shape in plan view of the binding portion 40B may be rectangle (square) or parallelogram. When binding the main-body second end 2E2 to the binding portion 40B for placement, it is a general practice to align the main-body second end 2E2 along the binding portion 40B. The user can place the disposable diaper for pet 1B of modified example 2 so as to unconsciously shrink it towards the head side, by aligning the main-body second end 2E2 along the binding portion 40B.

FIG. 7(c) illustrates a disposable diaper for pet 1C of modified example 3. The disposable diaper for pet 1C of modified example 3 has a mark portion 50C that is formed of a binding portion 40C, and the binding portion 40C has a head-side binding portion 40C1 composing a head-side mark portion 53C and a crotch-side binding portion 40C2 composing a crotch-side mark portion 54C. The head-side binding portion 40C1 and the crotch-side binding portion 40C2 are arranged in plan view with a space in between. The head-side binding portion 40C1 and the crotch-side binding portion 40C2 may only be apart, allowing arrangement with a space in between in the waistline direction W, or arrangement with a space in between in the cross direction Z. Each of the head-side binding portion 40C1 and the crotch-side binding portion 40C2 of modified example 3 extends in the cross direction Z, and the center in the waistline direction W of the head-side binding portion 40C1 resides on the center 2WC side in the waistline direction W of the main body portion 2, than the center in the waistline direction W of the crotch-side binding portion 40C2. With the head-side binding portion 40C1 and the crotch-side binding portion 40C2 arranged with a space in between, the user can stepwise bind the main-body second end 2E2, as compared with a structure having the head-side binding portion and the crotch-side binding portion integrated therein. Hence, one binding portion may be temporarily bound with the other binding portion being regularly bound, and then the one binding portion may be unbound and then re-bound. According to this mode of binding, the disposable diaper may easily be placed in a well-placed state on the body with improved fitting quality, as compared with the mode where the binding portion is bound in a single operation. This mode of binding also allows women or children with small hands to easily place the disposable diaper just as they intended, and can improve the fitting quality.

FIG. 8(a) illustrates a disposable diaper for pet 1D of a modified example 4. A mark portion 50D of the disposable diaper for pet 1D of modified example 4 is formed of an outer edge 30E1 on the main-body first end 2E1 side of the absorbent core 30. The outer edge 30E1 on the main-body first end 2E1 side of the absorbent core 30 is an outer edge in the waistline direction of the absorbent core 30, and inclines towards the inside in the waistline direction W, when viewed from the main-body fourth end 2E4 towards the main-body third end 2E3. According to the disposable diaper for pet 1D of modified example 4, the user will become easier to recognize a position of the outer edge of the absorbent core 30, in the process of alignment referring to the mark portion 50D for placement. By recognizing the position of the outer edge of the absorbent core, the user can dispose the absorbent core 30 so as not to cause leakage.

FIG. 8(b) illustrates a disposable diaper for pet 1E of modified example 5. A mark portion 50E of the disposable diaper for pet 1E of Modified Example 5 is formed of an outer edge on the main-body second end 2E2 side of the main body portion 2. The outer edge on the main-body second end 2E2 side of the main body portion 2 inclines in the cross direction Z and the waistline direction W, and inclines towards the inside in the waistline direction W, when viewed from the main-body fourth end 2E4 towards the main-body third end 2E3. According to the disposable diaper for pet 1E of Modified Example 5, the user can intuitively recognize the position and angle of the outer edge in the waistline direction of the main-body second end in the wearing state, enabling placement at a proper position and angle.

FIG. 8(c) illustrates a disposable diaper for pet 1F of Modified Example 6. A mark portion 50F of the disposable diaper for pet 1F of Modified Example 6 indicates an angle formed between the outer edge in the waistline direction W of the main-body first end 2E1 in the wearing state, and the outer edge in the waistline direction W of the main-body second end 2E2. According to the disposable diaper for pet 1F of Modified Example 6, the user can intuitively recognize the position and angle of the outer edge in the waistline direction of the main body in the wearing state, enabling placement at a proper position and angle.

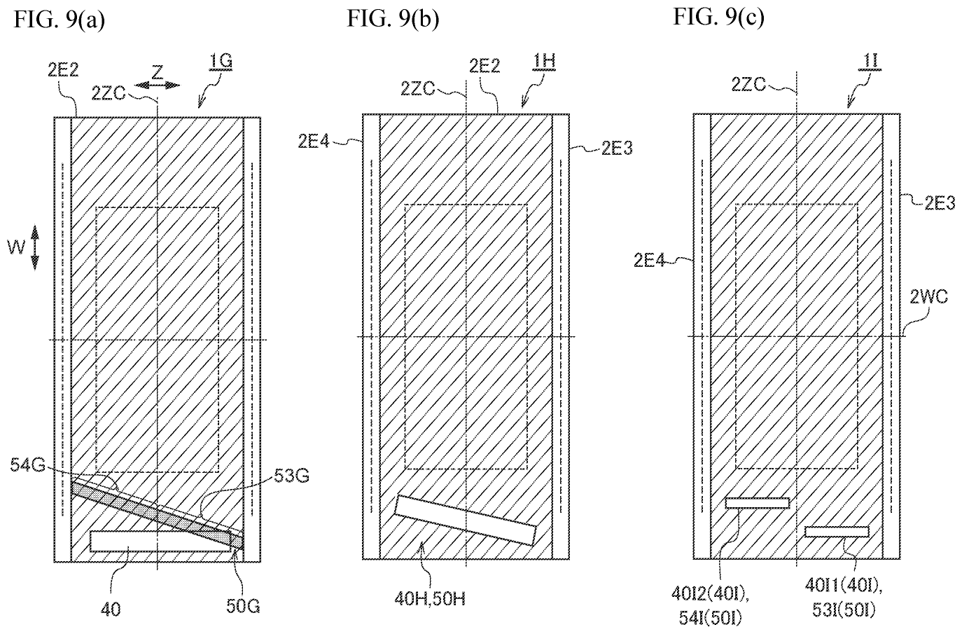

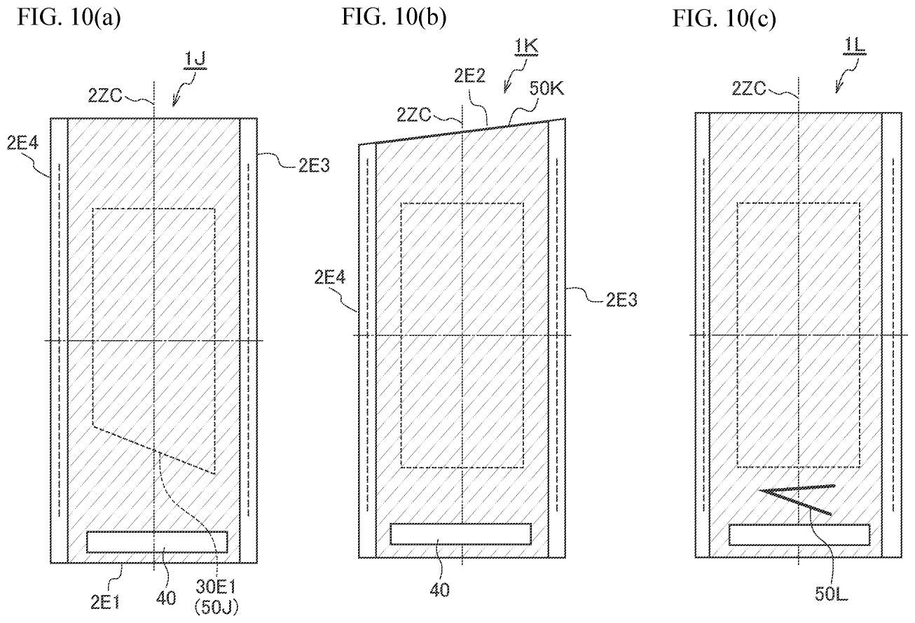

FIGS. 9(a) to 9(c) and 10(a) to 10(c) are plan views illustrating modified examples of the disposable diaper for pet of one or more embodiments, viewed from the non-skin contact side of the disposable diaper for pet. FIG. 9(a) illustrates a disposable diaper for pet 1G of Modified Example 7. A mark portion 50G of the disposable diaper for pet 1G of Modified Example 7, and the mark portion 50A of the disposable diaper for pet 1A of Modified Example 1 are axisymmetric about the center 2ZC in the cross direction Z of the main body portion 2. In the disposable diaper for pet 1G of Modified Example 7, at least either of a head-side mark portion 53G or a crotch-side mark portion 54G is arranged in an area that overlaps the binding portion 40. The head-side mark portion 53G resides outside, in the waistline direction, of the crotch-side mark portion 54G.

FIG. 9(b) illustrates a disposable diaper for pet 1H of Modified Example 8. A mark portion 50H of the disposable diaper for pet 1H of Modified Example 8, and the mark portion 50B of the disposable diaper for pet 1B of Modified Example 2 are axisymmetric about the center 2ZC in the cross direction Z of the main body portion 2. The mark portion 50H of the disposable diaper for pet 1H of Modified Example 8 is formed of a binding portion 40H. The edge in the waistline direction of the binding portion 40H in Modified Example 8 inclines towards the inside in the waistline direction, when viewed from the main-body third end 2E3 towards the main-body fourth end 2E4.

FIG. 9(c) illustrates a disposable diaper for pet 1I of Modified Example 9. A mark portion 501 of the disposable diaper for pet 1I of Modified Example 9, and the mark portion 50C of the disposable diaper for pet 1C of Modified Example 3 are axisymmetric about the center 2ZC in the cross direction Z of the main body portion 2. The disposable diaper for pet 1I of modified example 9 has a mark portion 50I that is formed of a binding portion 40I, and the binding portion 40I has a head-side binding portion 40I1 composing a head-side mark portion 53I and a crotch-side binding portion 40I2 composing a crotch-side mark portion 54I. The head-side binding portion 40I1 and the crotch-side binding portion 40I2 are arranged in plan view with a space in between. Each of the head-side binding portion 40I1 and the crotch-side binding portion 40I2 of modified example 9 extends in the cross direction Z, and the center in the waistline direction W of the head-side binding portion 40I1 resides more outward in the waistline direction W of the main body portion 2, than the center in the waistline direction W of the crotch-side binding portion 40I2.

FIG. 10(a) illustrates a disposable diaper for pet 1J of Modified Example 10. A mark portion 50J of the disposable diaper for pet 1J of Modified Example 10, and the mark portion 50D of the disposable diaper for pet 1D of Modified Example 4 are axisymmetric about the center 2ZC in the cross direction Z of the main body portion 2. The mark portion 50J of the disposable diaper for pet 1J of Modified Example 10 is formed of the outer edge 30E1 on the main-body first end 2E1 side of the absorbent core 30. The outer edge 30E1 on the main-body first end 2E1 side of the absorbent core 30 is an outer edge in the waistline direction of the absorbent core 30, and inclines towards the inside in the waistline direction W, when viewed from the main-body third end 2E3 towards the main-body fourth end 2E4.

FIG. 10(b) illustrates a disposable diaper for pet 1K of Modified Example 11. A mark portion 50K of the disposable diaper for pet 1K of Modified Example 11, and the mark portion 50E of the disposable diaper for pet 1E of Modified Example 5 are axisymmetric about the center 2ZC in the cross direction Z of the main body portion 2. The mark portion 50K of the disposable diaper for pet 1K of Modified Example 11 is formed of an outer edge on the main-body second end 2E2 side of the main body portion 2. The outer edge on the main-body second end 2E2 side of the main body portion 2 inclines in the cross direction Z and the waistline direction W, and inclines towards the inside in the waistline direction W, when viewed from the main-body third end 2E3 towards the main-body fourth end 2E4.

FIG. 10(c) illustrates a disposable diaper for pet 1L of Modified Example 12. A mark portion 50L of the disposable diaper for pet 1L of Modified Example 12, and the mark portion 50F of the disposable diaper for pet 1F of Modified Example 6 are axisymmetric about the center 2ZC in the cross direction Z of the main body portion 2. The mark portion 50L of the disposable diaper for pet 1L of Modified Example 12 indicates an angle formed between the outer edge in the waistline direction W of the main-body first end 2E1 in the wearing state, and the outer edge in the waistline direction W of the main-body second end 2E2.

Next, an exemplary method for manufacturing the thus designed disposable diaper for pet will be explained. The method for manufacturing the disposable diaper for pet may include:

a sheet feeding step of feeding a continuous sheet having sheet components arrayed therein;
a marking step of giving a mark portion by printing on the continuous sheet to provide the mark portion 50; and
an absorbent core laminating step of laminating the absorbent core 30 on the continuous sheet having the mark portion 50.

The sheet feeding step, feeding the continuous sheet having sheet components arrayed therein, takes part in feeding of the continuous sheet in which sheet components, composing any one of the topsheet 10, backsheet and core wrap, are arrayed. The marking step takes part in giving the mark portion 50 on the continuous sheet. The mark portion 50 may be given by printing, or by embossing. The core laminating step takes part in detection of the mark portion 50 on the continuous sheet. The mark portion 50 may be detected by detecting a position thereof using an image capturing means such as camera. The core laminating step takes part in adjustment of a position of lamination of the absorbent core 30, referring to the position of the mark portion 50. Adjustment of the position of lamination of the absorbent core 30 means adjustment of the positional relation between the absorbent core 30 and the continuous sheet. More specifically, the positional relation between the continuous sheet and the absorbent core 30 is adjustable by controlling the rate of feeding of the continuous sheet. Alternatively, an adjustment mechanism may be arranged adjacent to the sides, in a cross direction orthogonal to the feeding direction, of the continuous sheet, and a position in the orthogonal direction of the continuous sheet may be adjusted using such adjustment mechanism. With such method for manufacturing, the positional relation between the mark portion 50 and the absorbent core 30 may be kept constant. Hence, the disposable diaper when placed referring to the mark portion 50 will have the absorbent core 30 whose placement position is prevented from being misaligned one by one. With suppression of shift of the mark portion 50 over the whole range of the disposable diaper, alignment referring to such mark portion 50 will be enabled with improved accuracy.

Next, a packaged article having a plurality of disposable diapers for pet enclosed therein will be explained. The packaged article includes a plurality of disposable diapers for pet, and a wrapper for enclosing the plurality of disposable diapers for pet. The plurality of disposable diapers for pet includes a first disposable diaper having the first decoration part printed on the backsheet, and a second disposable diaper having the second decoration part designed differently from the first decoration part printed on the backsheet. The first decoration part and the second decoration part may only have design different from each other, and may be different from the first decoration part and the second decoration part in one or more embodiments. The disposable diaper in one or more embodiments has the first decoration part or the second decoration part, printed on the back film of the backsheet. With such first disposable diaper having the first decoration part and the mark portion, and the second disposable diaper having the second decoration part and the mark portion, one or more embodiments will have enhanced decorativeness, thereby can attract attention of the user, and can improve recognizability of the mark portion. The first decoration part and the second decoration part are parts having a design other the mark portion, and may have not only picture or pattern, but also background color solely represented by color.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

According to one or more embodiments of the present invention, it is possible to provide a disposable diaper for pet, which can be placed conforming to the waist shape of pet, and can improve the fitting quality.

### REFERENCE SIGNS LIST

1,1X,1B,1C,1D,1E,1F: Disposable diaper for pet
2: Main body portion
2E1: main-body first end
2E2: main-body second end
2E3: main-body third end
2E4:main-body fourth end
2P: inner face
2Q: outer face
10: Topsheet
20: Backsheet
21: back film
22: nonwoven back fabric
30: absorbent core
40,40B,40C: Binding portion
50,50X,50A,50B,50C,50D,50E,50F,50G,50H,50I,50J,50K,50L: Mark portions
53,53X,53A: head-side mark portion
54,54X,54A: crotch-side mark portion
T: thickness direction
W: Waistline direction
Z: Cross direction

What is claimed is:

1. A disposable diaper for a pet, comprising:
a main body comprising a topsheet, a backsheet, and an absorbent core between the top sheet and the backsheet; and
a binding portion that binds opposing parts of the main body when the disposable diaper is wrapped around a waist of the pet in a wearing state, wherein
the main body further comprises:
a main-body first end at one end in a waistline direction of the disposable diaper;
a main-body second end at another end in the waistline direction opposite to the main-body first end and that is on an outside of the disposable diaper in the wearing state;
a main-body third end at one end in a cross direction of the disposable diaper and that is on a head-side of the pet in the wearing state, wherein the cross direction is orthogonal to the waistline direction;
a main-body fourth end at another end in the cross direction opposite to the main-body third end and that is on a crotch-side of the pet in the wearing state; and
a mark portion, visible from a side of the backsheet, that indicates a position of the main-body second end in the wearing state,
the mark portion comprises:
a head-side mark portion disposed on one side from a center of the disposable diaper in the cross direction toward the main-body third end; and
a crotch-side mark portion disposed on another side from the center of the disposable diaper in the cross direction toward the main-body fourth end,
the head-side mark portion is closer to a center of the disposable diaper in the waistline direction than the crotch-side mark portion.

2. A disposable diaper for a pet, comprising:
a main body comprising a topsheet, a backsheet, and an absorbent core between the top sheet and the backsheet; and
a binding portion that binds opposing parts of the main body when the disposable diaper is wrapped around a waist of the pet in a wearing state, wherein
the main body further comprises:
a main-body first end at one end in a waistline direction of the disposable diaper;
a main-body second end at another end in the waistline direction opposite to the main-body first end and that is on an outside of the disposable diaper in the wearing state;
a main-body third end at one end in a cross direction of the disposable diaper and that is on a head-side of the pet in the wearing state, wherein the cross direction is orthogonal to the waistline direction;
a main-body fourth end at another end in the cross direction opposite to the main-body third end and that is on a crotch-side of the pet in the wearing state; and
a mark portion, visible from a side of the backsheet, that indicates a position of the main-body second end in the wearing state,
the mark portion comprises:
a head-side mark portion disposed on one side from a center of the disposable diaper in the cross direction toward the main-body third end; and
a crotch-side mark portion disposed on another side from the center of the disposable diaper in the cross direction toward the main-body fourth end,
the head-side mark portion is farther from a center of the disposable diaper in the waistline direction than the crotch-side mark portion.

3. The disposable diaper according to claim 1, wherein
the binding portion is:
on a face of a backsheet side of the main-body first end; and
bound to a face of a topsheet side of the main-body second end in the wearing state, and
one of the head-side mark portion or crotch-side mark portion is in a region adjacent to the binding portion.

4. The disposable diaper according to claim 1, wherein
the binding portion is:
on a face of a backsheet side of the main-body first end, and
bound to a side of a topsheet side of the main-body second end in the wearing state, and
one of the head-side mark portion or crotch-side mark portion is in a region overlapping the binding portion.

5. The disposable diaper according to claim 1, wherein, in the wearing state, one of the head-side mark portion or the crotch-side mark portion indicates a position of an outer edge of the main-body second end.

6. The disposable diaper according to claim 1, wherein
the binding portion is on the main-body first end, and
the binding portion is the mark portion.

7. The disposable diaper for pet according to claim 6, wherein
the binding portion comprises:
a head-side binding portion that is the head-side mark portion; and
a crotch-side binding portion that is the crotch-side mark portion, and
the head-side binding portion and the crotch-side binding portion are spaced apart in the waistline direction.

8. The disposable diaper according to claim 1, wherein the head-side mark portion and the crotch-side mark portion are connected and extend obliquely in the cross direction.

9. The disposable diaper according to claim 1, wherein the mark portion is an outer edge of the absorbent core on a side of the absorbent core closer to the main-body first end.

10. A method for manufacturing a disposable diaper for a pet, the method comprising:
feeding a continuous sheet arrayed with sheet components;
disposing a mark portion on the continuous sheet;
laminating an absorbent core on the continuous sheet with the mark portion; and
detecting, during the laminating, a position of the mark portion on the continuous sheet; and
adjusting a position of lamination of the absorbent core based on the position of the mark portion.

11. The disposable diaper according to claim 2, wherein
the binding portion is:
on a face of a backsheet side of the main-body first end, and
bound to a face of a topsheet side of the main-body second end in the wearing state, and
one of the head-side mark portion or the crotch-side mark portion is in a region adjacent to the binding portion.

12. The disposable diaper according to claim 2, wherein
the binding portion is:
on a face of a backsheet side of the main-body first end, and
bound to a side of a topsheet side of the main-body second end in the wearing state, and
one of the head-side mark portion or the crotch-side mark portion is in a region overlapping the binding portion.

13. The disposable diaper according to claim 2, wherein, in the wearing state, one of the head-side mark portion or the crotch-side mark portion indicates a position of an outer edge of the main-body second end.

14. The disposable diaper according to claim 2, wherein
the binding portion is on the main-body first end, and
the binding portion is the mark portion.

15. The disposable diaper for pet according to claim 14, wherein
the binding portion comprises:
a head-side binding portion that is the head-side mark portion; and
a crotch-side binding portion that is the crotch-side mark portion, and
the head-side binding portion and the crotch-side binding portion are spaced apart in the waistline direction.

16. The disposable diaper according to claim 2, wherein the head-side mark portion and the crotch-side mark portion are connected and extend obliquely in the cross direction.

17. The disposable diaper according to claim 2, wherein the mark portion is an outer edge of the absorbent core on a side of the absorbent core closer to the main-body first end.
